(19) United States
(12) Patent Application Publication
(10) Pub. No.: US 2023/0181352 A1
(43) Pub. Date: Jun. 15, 2023

(54) Systems, Methods and Apparatuses for Patient Temperature Control with Patient Heat Generation Detection

(71) Applicant: C. R. Bard, Inc., Franklin Lakes, NJ (US)

(72) Inventors: Marc E. Voorhees, Arvada, CO (US); Michelle Merrill, Marshfield, MA (US); Karine Rastatter, Hudson, CO (US)

(21) Appl. No.: 17/922,584

(22) PCT Filed: May 26, 2021
(86) PCT No.: PCT/US21/34308

§ 371 Date: Oct. 31, 2022

Related U.S. Application Data
(60) Provisional application No. 63/032,226, filed on May 29, 2020.

(51) Int. Cl. A61F 7/00

(52) U.S. Cl. CPC A61F 7/0085; A61F 2007/0054; A61F 2007/0093; A61F 2007/0095

(57) ABSTRACT

A patient temperature control system is disclosed that includes a heat exchange system configured to heat or cool a fluid, a circulating pump configured to circulate the fluid through the heat exchanger and at least one interconnectable pad and a control one or more processors and a non-transitory computer-readable medium having stored thereon logic. The logic, when executed by the one or more processors, causes operations including initiation of a targeted temperature management therapy, obtaining of measurements from fluid temperature sensors, a volumetric flow rate sensor and a patient temperature sensor, determining a level of patient heat generation based on one or more of the obtained measurements, and transmitting, via one or more wireless signals, data indicating the level of patient heat generation to a network device.

### PRIORITY

This application claims priority to U.S. Provisional Application No. 63/032,226, filed May 29, 2020, which is incorporated by reference in its entirety into this application.

### SUMMARY

Briefly summarized, embodiments disclosed herein are directed to systems, methods, and apparatuses for selectively controlling patient temperature. More particularly, aspects of the disclosure are directed to a targeted temperature management device that controls patient temperature through circulation of heated and/or cooled fluid through one or more interconnectable pads (“pads”) contacting a patient. Further, some embodiments of the disclosure are directed to systems, methods and apparatuses for measuring heat generated by a patient during targeted temperature management therapy.

Some embodiments of the disclosure describe a targeted temperature management (TTM) device that includes an electronic system configured to receive sensor measurements and cause a heater/chiller to automatically heat or cool a fluid. The TTM device may be configured to circulate the heated or cooled fluid through one or more pads in fluid communication with the TTM device. Further, the TTM device may be configured to receive temperature measurements of the water exiting the TTM device (flowing toward the pads) and of the water returning to the TTM device (returning from the pads). The electronic system may include logic that analyzes the water temperature measurements and volumetric flow rate of the fluid to determine an amount of heat transferred from the patient to the circulating fluid (which indicates an amount of patient heat generation). An indication of patient heat generation may be displayed via a graphical user interface on a display screen.

Some conventional TTM devices are known in the art to control (heat and/or cool) the temperature of a patient by circulating heated or cooled fluid through one or more heating/cooling components positioned directly or indirectly in contact with the patient's body. Examples of these heating/cooling components include external or surface cooling components, e.g., blankets, water blankets, wraps (corrugated), intervascular catheters configured to exchange heat with the blood stream, etc. Many of the conventional heating/cooling components are configured for circulation fluid throughout. Further, conventional TTM devices typically include one or more heat exchangers (e.g., heaters/chillers) for controlling the temperature of the fluid and one or more pumps that control circulation of heated or cooled fluid to the heating/cooling components. The fluid is then circulated through the heating/cooling components and returned to the TTM device (e.g., under positive or negative pressure). The temperature of the returned fluid may then be adjusted and re-circulated through the heating/cooling components to continue the targeted temperature management process in order to effectuate a target patient temperature.

As is understood, there are certain challenges that arise when providing TTM therapy to a patient. For instance, one common challenge clinicians face while providing TTM therapy is the existence, and specifically the detection, of patient shiver during TTM. Specifically, shiver causes an increase in metabolic rate and an increase in body temperature, which raises the difficulty in providing precise TTM therapy. In order to solve the shiver problem today, clinicians may increase the target temperature of the therapy. Alternatively, or in addition, clinicians may administer paralytic or sedative drugs to reduce shiver during TTM therapy. Yet another current solution to shiver include increasing the core temperature (i.e., increasing energy expenditure and heat generation), which adversely affects the TTM therapy. Further, in the current solutions, first clinicians need to identify shiver in patients undergoing hypothermia, which is a common technical challenge. Successful early identification and subsequent management of shivering facilitates the TTM therapy and outcome for the patient. Additionally, ineffective shiver management negates benefits associated with a cooling TTM therapy as well as increases systemic oxygen consumption by the patient. Conventionally, shiver detection has been a subjective assessment managed by clinicians. However, shivering in its initial state is often subtle enough that an observing clinician may not be aware of its occurrence.

Further, patient shiver is merely one manner in which a patient's body may generate heat that affects the TTM therapy. Other instances in which a patient may generate heat include, but are not limited to, development of a fever (e.g., infectious or viral), central fever (e.g., based on an injury to the interior hypothalamus, from trauma for example) and/or seizures. In particular, seizures are often a large concern with neonatal patients.

In view of the above, it should be understood that embodiments of the disclosure are applicable to all TTM therapies in which a patient may generate heat including both during a cooling therapy and a rewarming therapy and applicable to each instance in which patient heat generation occurs whether explicitly indicated in the examples above or otherwise.

Herein, the disclosure describes various embodiments of a system including a TTM device configured to detect patient heat generation during TTM therapy as discussed above. Additional embodiments are directed to a system including a TTM device configured to detect an amount of power utilized by the TTM device over time during a TTM therapy.

In one embodiment, a patient temperature control system is disclosed that comprises a heat exchange system configured to heat or cool a fluid, a circulating pump configured to circulate the fluid through the heat exchanger and at least one interconnectable pad, and a control module including one or more fluid temperature sensors, a volumetric flow rate sensor, one or more processors and a non-transitory computer-readable medium having stored thereon logic. The logic, when executed by the one or more processors, causes operations including initiating a targeted temperature management (TTM) therapy, obtaining measurements from the one or more fluid temperature sensors indicating a first temperature when the fluid is flowing to the at least one interconnectable pad and a second temperature when the fluid is returning from the at least one interconnectable pad, obtaining a measurement of volumetric flow rate of the fluid during circulation from the volumetric flow rate sensor, obtaining one or more measurements of patient temperature from a patient temperature sensor, determining a level of patient heat generation based on one or more of the obtained measurements of fluid temperature, volumetric flow rate and the patient temperature sensor, and transmitting, via one or more wireless signals, data indicating the level of patient heat generation to a network device.

The control module may be configured to receive return signals from the network device, the return signals employable by the control module to modify the TTM therapy. Further, determining of the level of patient heat generation may include calculating an amount of heat transfer between the fluid and the patient based on a difference between the first temperature and the second temperature, the volumetric flow rate of the fluid, and a heat capacity of the fluid.

In some embodiments, the logic, when executed by the one or more processors, determines the level of patient heat generation based at least in part on the obtained one or more of measurements of patient temperature. In some instances, the control module may be configured to receive user input corresponding to a selection of a size of the at least one interconnectable pad utilized during the TTM therapy, and wherein the logic, when executed by the one or more processors, determines the level of patient heat generation based at least in part on the user input. Additionally, the control module may be configured to receive a signal following coupling of the at least one interconnectable pad with the control module employable to identify a size of the at least one interconnectable pad.

In some embodiments, the logic, when executed by the one or more processors, causes further operations including generate a user interface display configured to visually indicate the level of patient heat generation. In some embodiments, the user interface display is rendered on a display screen of the control module and includes a time-based graphic providing an indication of patient heat generation. Further, in some embodiments, the time-based graphic provides the indication of patient generation throughout the duration of the TTM therapy following a wait-period following initiation of the TTM therapy. In other embodiments, the user interface is rendered on a display screen of the network device. In some embodiments, the level of patient heat generation is utilized as feedback to the logic of the control module and employable to generate one or more instructions to modify operation of the heat exchange system.

Additionally, embodiments of the disclosure include a method comprising initiating a targeted temperature management (TTM) therapy with a patient temperature control system including a heat exchange system configured to heat or cool a fluid and a circulating pump configured to circulate the fluid through the heat exchanger and at least one interconnectable pad, obtaining measurements from one or more fluid temperature sensors indicating a first temperature when the fluid is flowing to the at least one interconnectable pad and a second temperature when the fluid is returning from the at least one interconnectable pad, obtaining a measurement of volumetric flow rate of the fluid during circulation from the volumetric flow rate sensor, obtaining one or more measurements of patient temperature from a patient temperature sensor, determining a level of patient heat generation based on one or more of the obtained measurements of fluid temperature, volumetric flow rate and the patient temperature sensor, and transmitting, via one or more wireless signals, data indicating the level of patient heat generation to a network device.

In some embodiments, the patient temperature control system includes a control module including the one or more fluid temperature sensors, the volumetric flow rate sensor, one or more processors and a non-transitory computer-readable medium having stored thereon logic that is executable by the one or more processors. The patient temperature control system may be configured to receive return signals from the network device, the return signals employable by the patient temperature control system to modify the TTM therapy.

The method may also include determining the level of patient heat generation is based at least in part on the obtained measurements of fluid temperature and volumetric flow rate. Additionally, in some embodiments, determining the level of patient heat generation includes calculating an amount of heat transfer between the fluid and the patient based on a difference between the first temperature and the second temperature, the volumetric flow rate of the fluid, and a heat capacity of the fluid. In further embodiments, determining the level of patient heat generation is based at least in part on the obtained one or more of measurements patient temperature.

In some embodiments, the method further includes receiving user input, via the patient temperature control system, corresponding to a selection of a size of the at least one interconnectable pad utilized during the TTM therapy, and wherein determining the level of patient heat generation is based at least in part on the user input. The method may further include generating a user interface display configured to visually indicate the level of patient heat generation via a time-based graphic. In some instances, the method includes causing rendering of the user interface to be rendered on a display screen of the network device.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 illustrates a block diagram of a targeted temperature management (TTM) system according to some embodiments;

FIG. 2 illustrates a block diagram of the control module of the TTM system of FIG. 1 according to some embodiments;

FIG. 3 is a diagrammatic view of an exemplary use of the present invention according to some embodiments;

FIG. 4 is a logical representation of the control module of FIG. 2 according to some embodiments;

FIG. 5 is a logical representation of logic configured to control aspects of the TTM system of FIG. 1 deployed on an application of a network device according to some embodiments;

FIG. 6 illustrates an example user interface display directed to pad selection employable in conjunction with implementations of the TTM system of FIG. 1 according to some embodiments;

FIGS. 7A-7B illustrate example user interface displays directed to hypothermia and normothermia metrics employable in conjunction with implementations of the TTM system of FIG. 1 according to some embodiments;

FIG. 8 illustrates a flowchart illustrating an exemplary method of performing patient heat generation detection with the TTM system of FIG. 1 during TTM therapy according to some embodiments;

FIG. 9 illustrates a flowchart illustrating a detailed exemplary method of performing patient heat generation detection with the TTM system of FIG. 1 during TTM therapy according to some embodiments; and

FIG. 10 illustrates an example user interface display directed to patient heat generation employable in conjunction with implementations of the TTM system of FIG. 1 according to some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

### Targeted Temperature Management (TTM) Device

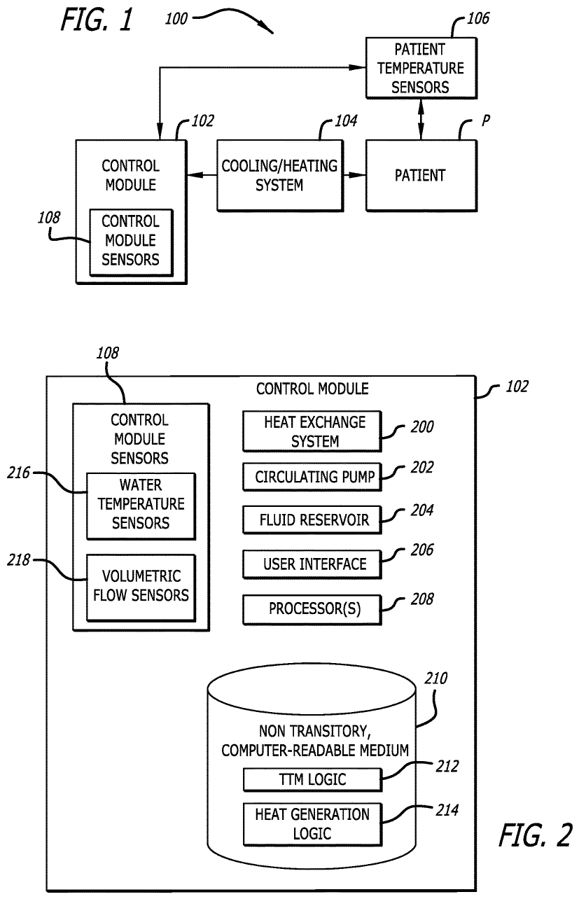

Referring now to FIG. 1, a block diagram of a targeted temperature management (TTM) system is shown according to some embodiments. As shown, a targeted temperature management (TTM) system 100 includes a control module 102, a cooling/heating system 104, patient temperature sensor(s) 106. The control module 102 is shown as being configured with control module sensors 108 to obtain various measurements such as of, for example, fluid temperature and/or volumetric flow of a fluid circulating between the control module 102 and the cooling/heating system 104 during TTM therapy.

For instance, the TTM therapy may include therapeutic hypothermia that is induced by the cooling/heating system 104. The cooling/heating system 104 may comprise any of a number of different modalities for selective cooling of a patient, including for example cooled contact pads, vascular cooling, patient emersion approaches and/or other systems for rapidly cooling a patient, e.g. systems as described in U.S. Pat. Nos. 6,669,715, 6,827,728, 6,375,674, 6,645,232, and WO 2007/121480, each of which is incorporated by reference in its entirety into this application.

As will be discussed in further detail below, the control module 102 includes logic and processor(s)/circuitry that, upon execution and processing, may be configured to analyze signals provided by any of the patient temperature sensors 106 and/or the control module sensors 108. For example, the patient temperature sensors 106 may provide the control module 102 with signals indicating a temperature of the patient P. The control module 102 may in turn alter, or maintain, the cooling/heating efforts of the cooling/heating system 104. In one embodiment, the cooling/heating efforts may refer to the temperature of fluid circulating from the control module 102 through the cooling/heating system 104 (e.g., contact pads or a catheter). Herein, the term “pads” will be used to refer to the cooling/heating system 104; however, such is not intended to be limiting of other possible embodiments of the cooling/heating system 104.

Additionally, the signals provided by either the patient temperature sensors 106 and/or the control module sensors 108 may be utilized to provide a visual and/or audible output of metrics or other status information of the patient and/or the TTM therapy via a user interface of the control module 102 or other output device (e.g. one or more light emitting diodes (LEDs)) and/or a network device communicatively coupled to the control module 102. In some embodiments, as will be discussed below, the output provides an indication of a magnitude, degree and/or stage of patient heat generation in response to the TTM therapy.

As may be appreciated, the processors/circuitry and a user interface of the control module 102 may be provided for interactive operations therebetween. More particularly, in conjunction with a given patient cooling procedure, a user may utilize the user interface to access and select a given one of a plurality of treatment/therapy protocols, e.g. corresponding with a given protocol established at a given user site (e.g. for a particular physician). For example, a user may select via user input to initiate a therapy of either Normothermia or Hypothermia (cooling or rewarming).

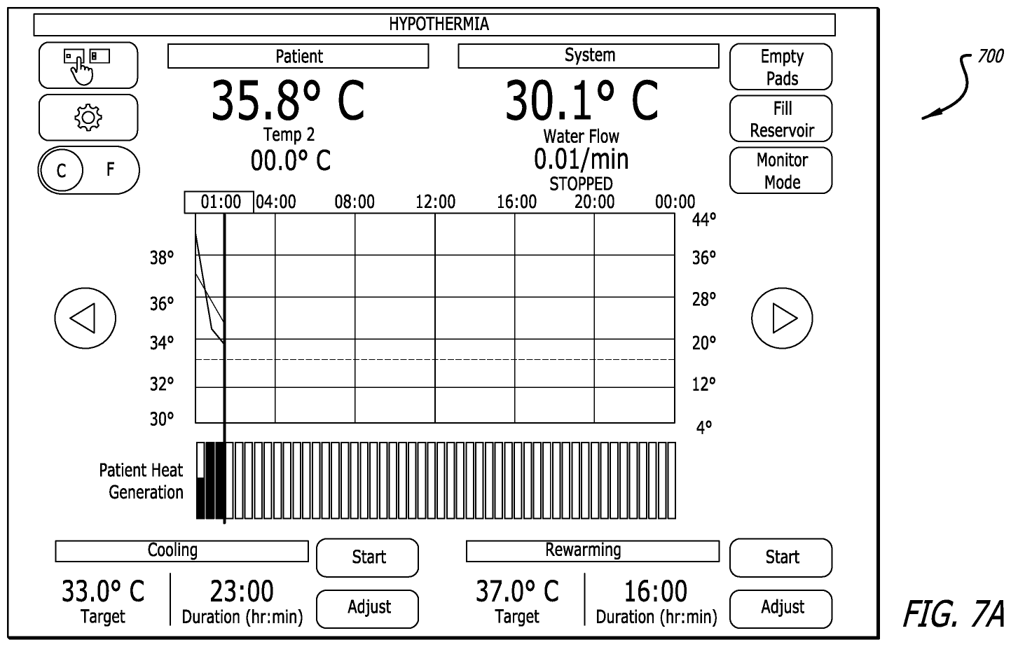

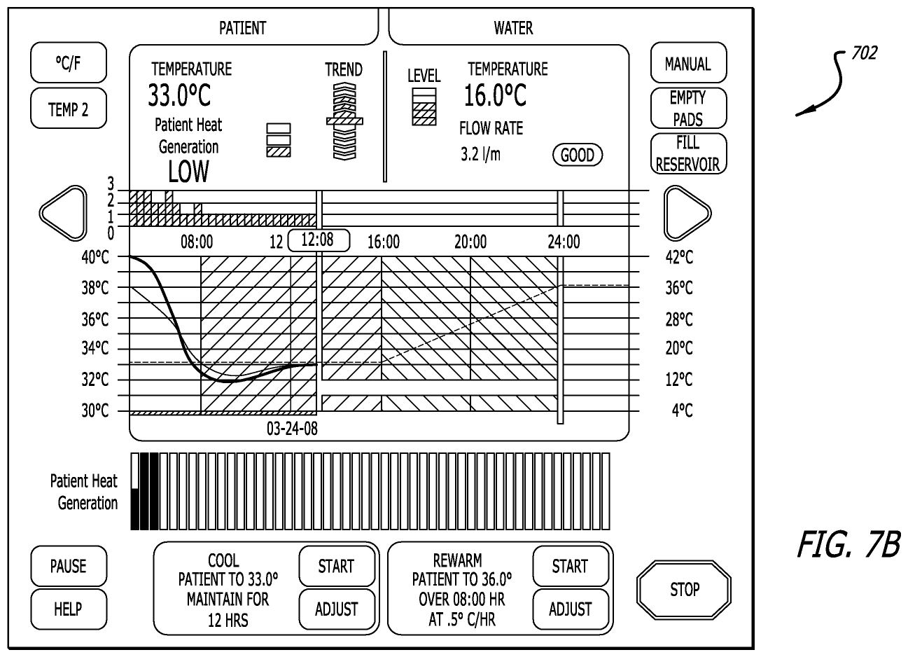

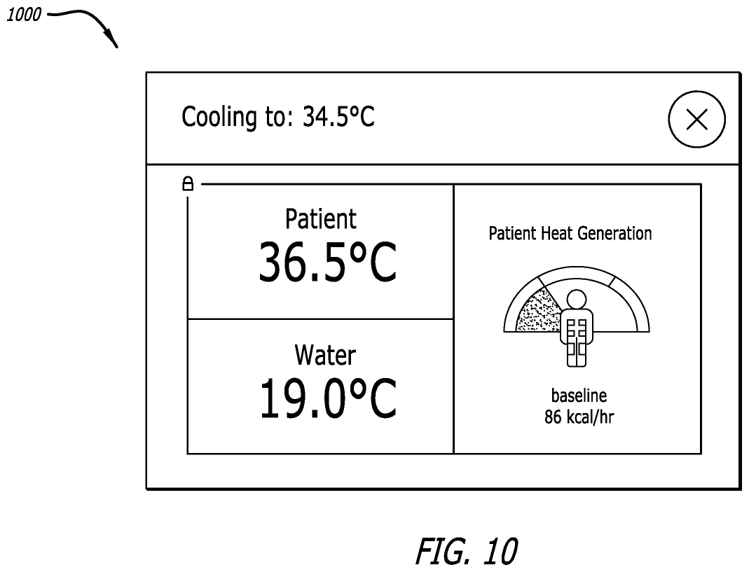

In turn, for a selected treatment/therapy, the control module sensors 108 may be configured to detect, obtain or otherwise measure variables pertaining to the provision of the patient cooling procedure. For instance, as will be discussed below, embodiments of the TTM system 100 include the control module sensors 108 being configured to measure the volumetric flow rate of the fluid circulating between the control module 102 and the pads 104 as well as the temperature of the circulating fluid at exit and entry points of the control module 102 (i.e., exiting the control module 102 to circulate through the pads 104, and returning to the control module 102 following circulation through the pads 104). Processors/circuitry of the control module 102 may receive the measurements as one or more signals such that logic, upon execution by the processors/circuitry, may determine the heat transferred between the patient P and the circulating fluid, and may also determine the heat generated by the patient P. Indications of the determination of patient heat generation may then be displayed via a user interface. Examples of such a display are shown in FIGS. 7A-7B and 10.

In some embodiments, user input received via one or more interactive display screens may be utilized during the determination by the TTM system of patient heat generation. For example, the size of the one or more pads utilized by as part of the TTM system 100 may affect or influence determinations made by the logic of the control module 102.

The signals and the user input may be stored in non-transitory computer-readable medium of the control module 102, or otherwise accessible thereby. By way of example, the above-noted analyses and determinations may include an algorithmic analysis as to patient heat generation in kcal/hour, the duration of patient heat generation and/or the patient heat generation on a time-scale basis associated with a given TTM therapy (e.g. collectively “trend data”). Ongoing treatment information and/or future anticipated treatment information as determined through such analyses or determinations may be provided to a user through user interfaces, wherein such further information is based in part on the trend data assessment.

Referring now to FIG. 2, a block diagram of the control module of the TTM system of FIG. 1 is shown according to some embodiments. The control module 102 is seen to include a plurality of components including at least a heat exchange system 200, a circulating pump 202, a fluid reservoir 204, a user interface 206, one or more processors (“processor”) 208, and non-transitory computer-readable medium 210 that has stored thereon TTM logic 212 and patient heat generation logic 214. The heat exchange system 200 is configured for affecting at least one of heating or cooling a fluid that circulates through the pads 104. The circulating pump 202 is configured to circulate the fluid through the heat exchange system 200 and the pads 104 to affect heat transfer between the pads 104 and the patient P.

The control module 102 may also include a fluid reservoir 204 that is fluidly interconnectable with the pads 104. The fluid reservoir 204 may be utilized to contain fluid that is removable from the reservoir to fill/circulate through the pads 104 during use. In conjunction with this aspect, the system may be defined so that, during normal heating/cooling operations, fluid is circulatable through the pads 204 and the heat exchange system 200 by the circulating pump 202. Additional embodiments have been contemplated in which the control module 102 includes a plurality of fluid reservoirs as described in U.S. Pat. No. 6,645,232, which is incorporated by reference in its entirety into this application.

Additionally, the control module 102 may include the user interface 206 as an output device for providing output information in at least one of an audible and visual form as described here. By way of example, the information may be provided via an interactive display (e.g., a touchscreen) and, optionally, physical input buttons.

The control module 102 may include the non-transitory computer-readable medium 210 that includes several logic modules, the operations of which are described herein. Generally, the TTM logic 212 includes logic, algorithms and/or data that, upon execution by the processor 208, cause performance of operations that instruct the heat exchange system 200 to warm or cool fluid circulating through the pads 104. In generating instructions for the heat exchange system 200, the TTM logic 212 may consider several parameters including, one or more of, profile information of the patient P (e.g., age, gender, weight, etc.), the number and size of pads 104 (and the extent to which the pads 104 cover the patient P), the signals received from the patient temperature sensors 106 and the control sensors 108, and any user input.

Similarly, the heat generation logic 214 includes logic, algorithms and/or data that, upon execution by the processor 208, cause performance of analyses, determinations and/or calculations to determine an amount of heat generated by a patient based on received signals from one or more sensors of the TTM system 100. In determining the amount of heat generated by the patient, the heat generation logic 214 may consider several parameters including, one or more of, measurements of fluid temperature (Tᵢₙ, Tₒᵤₜ), fluid density, mass flow rate of the fluid, heat capacity of the fluid, a size and number of the pads 104 utilized, etc. It should be noted that any of the determinations, assessments or analyses of the logic discussed herein may be provided or indicated via alerts, alarms or general displays via the user interface 206 or otherwise (e.g., other displays, speakers, or via communication of such to remote displays or speakers, such as of a network device, e.g., a mobile device, communicatively coupled to the control module 102).

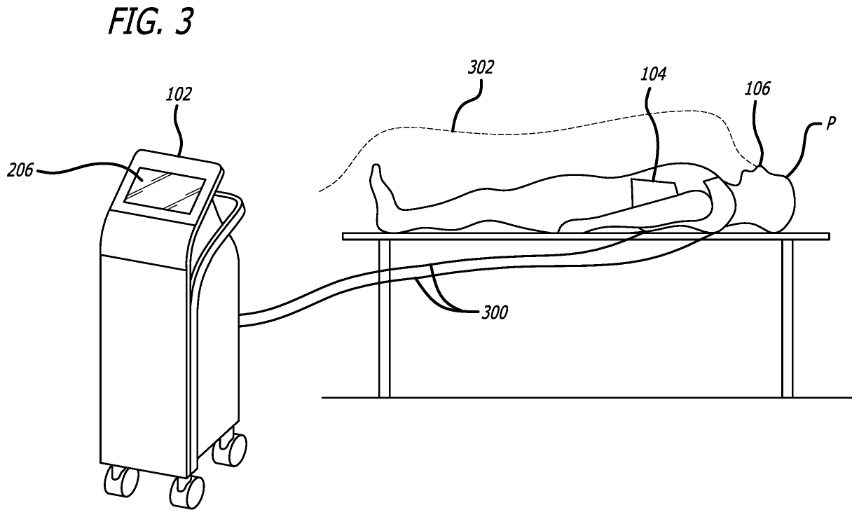

Referring now to FIG. 3, a diagrammatic view of an exemplary use of the present invention is shown according to some embodiments. FIG. 3 provides an example of one use of the TTM system 100 whereby the control module 102 is located proximate to the patient P. The patient P may be placed on a operating table, a hospital bed, or otherwise. Additionally, the control module 102 is shown to include the user interface 206 providing a user with ability to provide user input, which may cause initiation of execution of logic of the control module 102. The control module 102 may be in fluid communication with the pads 104 via fluid delivery lines 300 and may be communicatively coupled to the patient temperature sensor 106 via the wired connection 302. It should be understood that the communicatively coupling of the control module 102 and the patient temperature sensor may be via wireless connection.

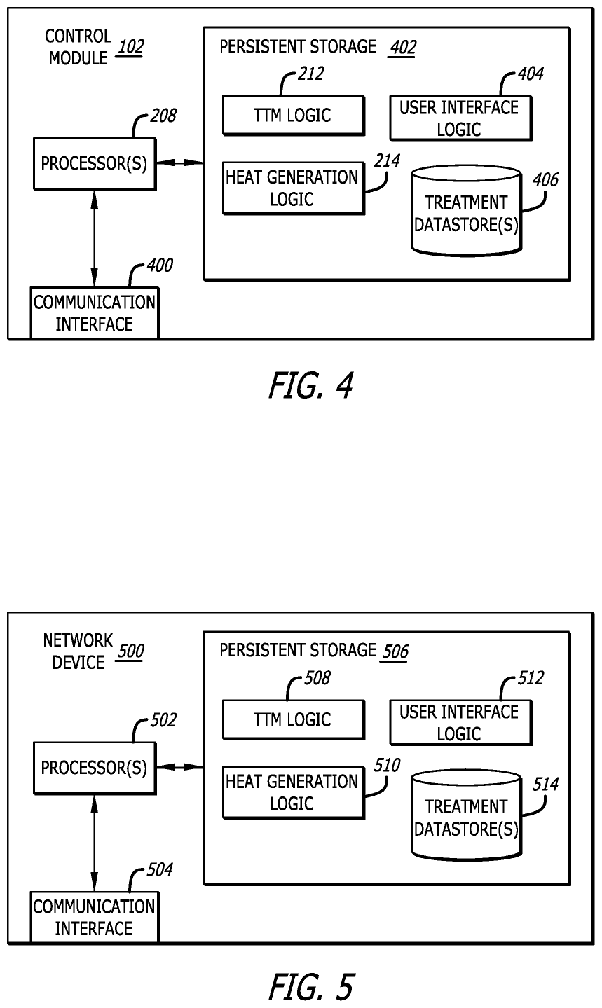

Referring now to FIG. 4, a logical representation of the control module of FIG. 2 is shown according to some embodiments. The control module 102 comprises one or more processors 208 that are coupled to the communication interface 400, which enables communication with other network devices, such as a clinician's mobile device and/or any of the sensors discussed in the disclosure. According to one embodiment of the disclosure, the communication interface 400 may be implemented as a physical interface including one or more ports for wired connectors. Additionally, or in the alternative, communication interface 400 may be implemented with one or more radio units for supporting wireless communications with other network devices.

The processor(s) 208 is further coupled to the persistent storage 402, e.g., non-transitory, computer-readable medium. According to one embodiment of the disclosure, the persistent storage 402 may include (i) the TTM logic 212, (ii) the heat generation logic 214, (iii) the user interface logic 404, and (iv) one or more treatment database(s) 406. Of course, when implemented as hardware, one or more of these logic units could be implemented separately from each other. As referenced above, the TTM logic 212 includes logic, algorithms and/or data that, upon execution by the processor 208, cause performance of operations that instruct the heat exchange system 200 to warm or cool fluid circulating through the pads 104. Further, the heat generation logic 214 includes logic, algorithms and/or data that, upon execution by the processor 208, cause performance of operations that analyze the signals received from at least the water temperature sensors 216, and optionally, the volumetric flow sensors 218, to determine heat generated by the patient P over a particular time period.

Referring to FIG. 5, a logical representation of logic configured to control aspects of the TTM system of FIG. 1 deployed on an application of network device 800 is shown according to some embodiments. The network device 500 comprises one or more processors 502 that are coupled to the communication interface 504, which enables communication with other network devices, such as the control module 102. According to one embodiment of the disclosure, the communication interface 504 may be implemented as a physical interface including one or more ports for wired connectors. Additionally, or in the alternative, communication interface 504 may be implemented with one or more radio units for supporting wireless communications with other network devices.

The processor(s) 502 is further coupled to the persistent storage 506, e.g., non-transitory, computer-readable medium. According to one embodiment of the disclosure, the persistent storage 506 may include (i) the TTM logic 508, (ii) the heat generation logic 510, (iii) the user interface logic 512, and (iv) one or more treatment database(s) 514. Of course, when implemented as hardware, one or more of these logic units could be implemented separately from each other. Each of the TTM logic 508 and the heat generation logic 510 may include all or a portion of the corresponding logic described above with respect to FIG. 2. For instance, each of the TTM logic 508 and the heat generation logic 510 may include logic that communicates with the corresponding logic stored on the control module 102 (e.g., the TTM logic 508 corresponds to the TTM logic 212) such that the logic stored on the network device 500 may initiate any procedure of the corresponding logic and/or receive the same signals received by the corresponding logic of the control module 102. In other embodiments, the logic stored on the network device 500 may merely receive signals from the control module 102 (e.g., potentially mirroring those received by the logic of the control module 102) and enable display of such information as a result of processing of the user interface logic 512. In order to initiate a procedure, the logic of the network device 500 may query the one or more treatment database 514, which may store preset procedures, protocols or other information relevant to initiating a TTM therapy.

In some embodiments, the network device 500 may be, for example, a mobile device (phone, tablet, etc.) of a medical professional that has installed and processing thereon certain logic modules (e.g., applications or “apps”) as discussed herein. These logic modules, e.g., the TTM logic 508, the heat generation logic 510 and/or the user interface logic 512, may receive wireless signals directly from sensors of the TTM system 100 including the patient temperature sensors 106 and/or the control module sensors 108, or indirectly via the control module 102. The logic 508-512 may then perform processing as discussed below with respect to the corresponding logic modules of the control module 102. The logic 508-512 may then perform operations causing signals to be transmitted to the control module 102 for alteration of the TTM therapy or rendering of user interface displays on the user interface 206. Additionally, the user interface logic 512 may cause rendering of illustrations on a user interface of the network device 500. Thus, the logic 508-512 enables a medical professional to monitor and, optionally control, the TTM therapy from a location remote to that of the patient P.

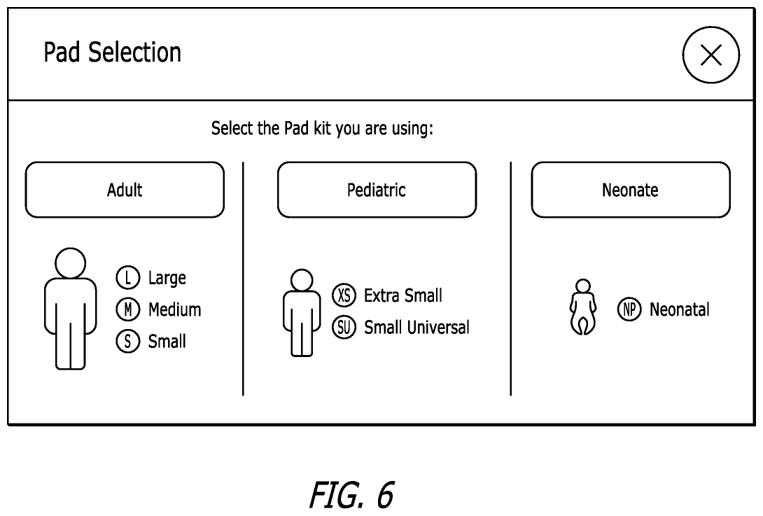

Referring to FIG. 6, an example user interface display directed to pad selection employable in conjunction with implementations of the TTM system of FIG. 1 is shown according to some embodiments. As referenced above, a TTM therapy may be selected via user input, e.g., Normothermia or Hypothermia (cooling or rewarming). Following the selection of the therapy to be provided, the logic of the control module 102, such as the TTM logic 212 and the user interface logic 404, may generate and cause the rendering of the user interface display 600 that is configured to received additional user input corresponding to selection of a size of pads to be used during the selected therapy. As an exemplary illustration, the user interface display 600 provides three categories from which a user may select one or more size options included therein: Adult 602 (Large, Medium, Small), Pediatric 604 (Extra Small, Small Universal) and Neonate 606 (Neonatal). Receipt of user input corresponding to a selection of a size of pads to be used will influence analyses, determinations and/or calculations performed by one or more of the TTM logic 212 and/or the heat generation logic 214. It should be understood that alternative, additional or fewer options may be presented for user selection via the user interface display 600.

Selection of the size of a pad to be used for TTM therapy may determine a calculation or analysis performed by the heat generation logic 214. The calculations utilized by each logic module will be discussed in further detail below.

Referring to FIGS. 7A-7B, example user interface displays directed to hypothermia and normothermia metrics employable in conjunction with implementations of the TTM system of FIG. 1 are shown according to some embodiments. As illustrated, various user interfaces may be generated and provided by the control module 102 that are configured to receive user input indicating information related to the circulation of cooled and/or warmed fluid through pads 104 to adjust a patient's temperature in accordance with a predetermined, automated and/or otherwise controllable protocol. Additionally, various user interfaces display information relating to an amount of heat generated by the patient P, such as relative amount displayed via a scale. In some embodiments, the scale may be a set of horizontally arranged icons (e.g., rectangles) that indicate a measure of time and may correspond to a time-based graph of patient/fluid temperature (illustrated in both interactive screen 700 of FIG. 7A and interactive screen 702 of FIG. 7B). Additionally, or in the alternative, other indicators may be displayed such as a textual indicator of the patient heat generation level or a series of blocks (e.g., three) indicating a current or most recent patient heat generation level, with each block corresponding to a level (both illustrated in FIG. 7A).

Further, as illustrated in FIG. 10, any of the exemplary illustrations shown in the figures may include a numerical indicator of the amount of heat transfer (W) or be converted and illustrated as the amount of patient heat generation (e.g., kcal/hour). It should be understood that various user interfaces may be configured and designed to display any of the information discussed in the disclosure on a single user interfaces or on separate user interfaces.

With respect to FIG. 7A, the interactive screen 700 may be provided at user interface 206 which includes a graphic display portion that graphically illustrates temperature-related data in a first region, e.g., as a function of time, and that further illustrates patient heat generation data as a function of time in a second region. The first region may present a first plot of a target patient temperature level as a function of time, e.g. a predetermined patient temperature adjustment rate plot reflecting a desired patient temperature to be reached by controlling the temperature of the circulated fluid. Further, a second plot of a measured patient temperature as a function of time may be presented. Additionally, a third plot of a measured temperature of the fluid circulated by the control module 102 though the pads 104 may be provided.

With respect to FIG. 7B, the interactive screen 702 provides an alternative example of the information of FIG. 7A displayed in a varied form. For example, as shown by FIG. 7B, a region of an interactive screen 702 may be provided to visually display patient heat generation data in relation to a predetermined magnitude scale. By way of example, a plurality of predetermined levels of patient heat generation may be graphically presented as a function of time. In the illustrated example, four levels of detected patient motion may be provided to a user, wherein no visual indication is provided for a low, or “zero” level of motion, and wherein increasing level of motions may be graphically presented by one, two or three stacked “box” indicators. Additionally, text indicating the level may accompany the graphic, e.g., “LOW” as seen in FIG. 7B.

As may be appreciated, by visually monitoring the level of patient heat generation displayed on the screens 700-702, medical personnel may assess the need and/or desirability for taking responsive action. For example, such responsive action may include the initiation of a warming procedure and/or a modification to the patient cooling/warming protocol discussed hereinabove (e.g. decreasing a target patient cooling rate and/or increasing targeted temperature for patient cooling). As discussed herein, the logic of the control module 102 may analyze received signals and automatically initiate such modifications, for example, when the signals received from the sensors 106-108 are processed by the logic of the control module 102 to indicate the patient heat generation level is above a predetermined threshold. It should be understood that FIGS. 7A-7B provide examples of the multiple of ways by which the information provided in the disclosure may be illustrated on a user interface.

### Patient Heat Generation Detection

Embodiments herein disclose a patient heat generation feature of the TTM system 100 of FIG. 1 that utilizes a heat transfer calculation to detect when patient heat generation is occurring. As energy expenditure from the patient increases, the amount of heat transferred from the patient to the TTM system 100 (and specifically to the circulating fluid) also increases.

Patient heat generation may be quantified using a hypermetabolic index (HMI). The HMI is comprised of a series values calculated from the ratio of energy expenditure during a shivering episode to resting energy expenditure. Specifically, the hypermetabolic index (HMI) for energy expenditure is derived by dividing the time-averaged measured value of resting energy expenditure (REE) (kcal/d) by expected energy expenditure (kcal/d). REE, or basal metabolic rate (BMR), is approximately 80 W (68.6 kcal/hr) for an average human. Expected energy expenditure values may be derived from the Harris-Benedict equation, which may be adjusted for patient acuity. Additionally, as is known in the art, the Bedside Shiver Assessment Scale (BSAS) quantifies patient energy expenditure during TTM therapy on a scale of 0-3. The BSAS values may be correlated with HMI values.

As discussed above, conventionally, a patient may be visually monitored by a medical professional. However, shiver—and in effect, patient heat generation—associated with certain HMI levels, e.g., 1.5-2.5 out of a range of 0-3, often goes unnoticed in patients receiving TTM therapy. Therefore, providing a system, method and apparatus that enables detection of HMI at various levels would be advantageous to users over conventional systems that detects patient heat generation at various HMI and BSAS levels and generates a display indicating patient heat generation. Such a system, method and apparatus would enable a user to adjust the TTM therapy as needed.

According to some embodiments of the disclosure, the heat generation logic 214 may receive signals during provision of a TTM therapy to a patient. For example, the heat generation logic 214 may receive a flow rate of fluid through the control module 102 and the pads 104 as well as temperature measurements of the fluid, e.g., (i) exiting the control module 102 and entering the delivery lines to circulate through the pads 104 (Tₒᵤₜ), and (ii) returning to the control module 102 via the delivery lines following circulation through the pads 104 (Tᵢₙ).

Based on the received signals, the heat generation logic 214 performs an analysis of the signals to determine patient heat generation. In some embodiments, the determination includes utilization of the heat generation calculation:

Q=m*C_{P}*ΔT Equation 1

Where: Q=heat transfer, m=mass flow rate of the fluid, C_{P}=heat capacity of the fluid, and ΔT=Tᵢₙ−Tₒᵤₜ. In some embodiments, a constant heat capacity of 4.18 kcal/kg-K is used. Additionally, the volumetric flow rate of the circulating fluid is measured by the volumetric flow sensors 218 and is converted by either the volumetric flow sensors 218 or the heat generation logic 214 to mass flow rate using a constant density of 1 kg/L.

Further, the user interface logic 404 may generate a display portion that graphically illustrates the resultant measure of heat transfer (W) on the user interface 206. In some instances, a numerical value may be displayed. However, in other instances, a graphical representation of a gauge illustrating a spectrum of heat transfer may be displayed where a “needle” or line indicates the resultant measure of heat transfers across the spectrum. In some embodiments, the spectrum has a range of 0-500 W. Further, the display may be color-coded based on the resultant measure of heat transfer. For example, the gauge may be displayed in a first color (e.g., blue) when the resultant measure of heat transfer is below a threshold and may be displayed in a second color (e.g., orange) when the resultant measure of heat transfer is above the threshold. It should be understood that multiple thresholds may be utilized with the gauge being displayed in a different for each threshold that is met or exceeded.

In other embodiments, Equation 1 may be adjusted to achieve heat transfer in units of kcal/hour to be:

$$Q ( \frac{kcal}{hour} ) = {60}^{*} {m ( \frac{L}{min} )}^{*} Δ T ( K or °C . ) \qquad Equation 2$$

Following determination of heat generation, the value may be stored in, for example, the heat generation datastore 408 of FIG. 4. In embodiments in which Equation 2 are utilized, the heat generation logic 214 may determine a level or threshold of patient heat generated based on the calculated

$$Q ( \frac{kcal}{hour} ) .$$

Exemplary levels or thresholds may be: Baseline: 0-100 kcal/hour; Low heat generation: 100-200 kcal/hour; and High heat generation: >200 kcal/hour. It should be understood that the above recited thresholds may be adjustable prior to the initiation of a TTM therapy or, in some instances, during provision of the TTM therapy. Further, alternative measurement scales may be utilized, e.g., instead of kcal/hour. For instance, kcal/day, kcal/week. Following determination of a heat generation level, the user interface logic 404 may generate a display portion that graphically illustrates the determined heat generation level. Additionally, the user interface logic 404 may generate a graphical representation of the patient's heat generation level over a given time period. Examples of such graphical representations are shown in FIGS. 7A-7B.

As discussed above and illustrated in FIG. 6, the user interface 206 of the control module 102 may receive user input corresponding to a selection of a size of a pad 104 to be used in the TTM therapy. In some embodiments, the Equations 1-2 discussed above may be utilized either as a default (e.g., when no user input indicating a pad size selection is received) and/or when the user input corresponds to selection of an Adult sized-pad (e.g., user input activating a button corresponding to selection of Adult 602 in FIG. 6).

In instances in which received user input corresponds to selection of either Pediatric 604 or Neonate 606 (e.g., a pad size smaller than would be required to cool an average-sized adult), alternatives from the application of Equations 1-2 may be used to determine the patient heat generation. Specifically, one or more predetermined rule sets may be applied when a pad size smaller than first size is used. In some embodiments, the predetermined rule sets may be stored on the non-transitory, computer-readable medium 210 of FIG. 2.

For instance, the patient heat generation determination may be made based on patient temperature trend data, which is based on prior patient temperature recorded during provision of the current TTM therapy, with the determination made in accordance with one or more predetermined rule sets. For example, in some embodiments, the following may be utilized to determine a patient heat generation level: (i) Baseline, when the patient temperature is below the target temperature or less than 0.1° C. above the target temperature, regardless of the patient temperature trend; (ii) Low heat generation, when the patient temperature is greater than or equal to 0.1° C. above the target temperature and the patient temperature trend is increasing at a rate of 0-0.25° C./hour; and (iii) High heat generation, when the patient temperature is greater than or equal to 0.1° C. above the target temperature and the patient temperature trend is increasing at a rate greater than 0.25° C./hour.

The discussions regarding wait times and the graphical representation (“indicator”) of the level of patient heat generation provided above apply equally with respect to the determinations of patient heat generation based regardless of pad size. Additionally, regardless of pad size (and hence equation used or determination method), the indicator may illustrate the patient heat generation level is at Baseline (or may not be displayed) when the patient temperature is trending down or below the target temperature.

In some embodiments, patient heat generation will be determined over a 5 minute moving average using one or more of the above discussed equations. Several additional parameters may affect or alter the display of the graphical representations discussed above. For example, in some embodiments, graphical representations associated with patient heat generation will not be displayed when TTM therapy has been stopped or paused. When the TTM therapy is stopped or paused and restarted, in some instances the heat generation logic 214 will return to continued determination of patient heat generation following a certain wait period, e.g., 15 minutes, which may serve to allow the TTM system 100 to stabilize, e.g., stabilize flow rate, and eliminate artifacts from stagnant water temperature being flushed through the TTM system 100. For example, the 15 minute period includes a 5 minute moving average, where the 5 minute moving average begins after 10 minutes of use following the target temperature change. In some embodiments, a graphical representation of a level of patient heat generation may be displayed in a grey color during the wait period. The graphical representation of the level of patient heat generation may be referred to as an “indicator.”

As the target temperature may be changed during the TTM therapy (e.g., via user input), the heat generation logic 214 may account for such. In some embodiments, the heat generation logic 214 will return to continued determination of patient heat generation following a certain wait period, e.g., 15 minutes. For example, the 15 minute period includes a 5 minute moving average, where the 5 minute moving average begins after 10 minutes of use following the target temperature change. In some embodiments, the indicator may be displayed in a grey color during the wait period. In some embodiments, the wait period discussed above with respect to a target temperature change may not apply dining a patient rewarm phase of the TTM therapy.

Further, the user interface logic 404 in conjunction with the heat generation logic 214 may cause the indicator to display an indication that the patient heat generation is at Baseline when patient temperature is trending down (e.g., over a 5 minute time period) or is below the target temperature.

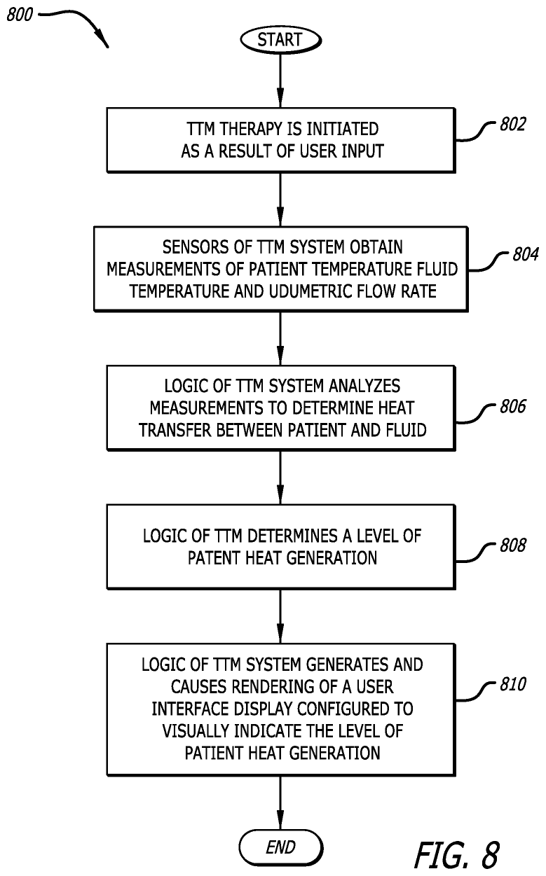

Referring now to FIG. 8, a flowchart illustrating an exemplary method of performing patient heat generation detection with the TTM system of FIG. 1 during TTM therapy is shown according to some embodiments. Each block illustrated in FIG. 8 represents an operation performed in the method 800 of providing TTM therapy using the TTM system 100. Herein, the method 800 starts with the initiation of a TTM therapy in response to receive user input (block 802). For instance, the TTM therapy may be directed at cooling a patient or rewarming a patient from a state of hypothermia. Following initiation of the TTM therapy, the control module 102 of the TTM system 100 receives data (signals) from the sensors 108 on an ongoing basis (e.g., such as at periodic or aperiodic intervals) (block 804). In some embodiments, the received data corresponds to fluid temperature measurements and volumetric flow rate measurements obtained by the sensors 108.

In response to receipt of the sensor data, logic of the TTM system 100 performs one or more analyses, determinations and/or calculations resulting in a determination of a level of heat transfer between the fluid circulating through the TTM system 100 (specifically through the pads 104) and the patient (e.g., patient heat generation) (block 808). Following determination of the level of patient heat generation, logic of the TTM system 100 generates and causes rendering of a user interface display configured to visually indicate the level of patient heat generation (block 810).

The method 800 may further include providing the above-noted heat transfer (W) or the patient heat generation (e.g., kcal/hour) to logic of the control module 102 as feedback, e.g., as an electrical data signal. The logic, such as the TTM logic 212, may utilize the amount of heat transfer or patient heat generation in the automated control of the fluid temperature. For example, the TTM logic 212 includes algorithms and/or data that, upon execution by the processor 208, cause performance of operations that instruct the heat exchange system 200 to warm or cool fluid circulating through the pads 104. Thus, such algorithms may receive the amount of heat transfers or patient heat generation as an input that affects the instructions for the heat exchange system 200 (e.g., when patient heat generation is detected above certain thresholds, the instructions may be to increase the fluid temperature by a calculated amount to slow the cooling of the patient's body in order to reduce the amount of patient heat generation). Alternatively, or in addition, the amount of heat transfer or patient heat generation may be provided to the user interface logic 404. In some embodiments, the user interface logic 404 is triggered to provide an alert, alarm or other notice to a user of the level of the patient heat generation in addition to providing one or more illustrations as discussed herein. For example, an audio alert accompanied by a visual display box rendered on the user interface 206 may be generated when the amount of heat transfer or patient heat generation is above a predetermined threshold. Other examples may include the automated transmission of an electronic alert (text, email, automated phone call, etc.) to an electronic device of one or more users (e.g., medical professionals supervising the TTM therapy).

In some embodiments, the logic of the control module 102 may refer to a proportional—integral—derivative (PID) controller. Operation of the the PID controller includes one or more of the measured water temperatures as a feedback signal, which is compared with the target temperature and fed to the PID algorithm. The PID algorithm determines any necessary adjustments to the water temperature (Tₒᵤₜ) that is proportional to the change delta between the measured temperature signals taking into account a time factor in bringing the patient to the target temperature. The proportional, integral and derivative controls helps the unit to automatically compensate for changes in the water temperature thereby providing accurate and stable control of the water temperature (Tₒᵤₜ). In some embodiments, the PID algorithm may also be provided the patient heat generation (as discussed above with respect to either Equations 1-2).

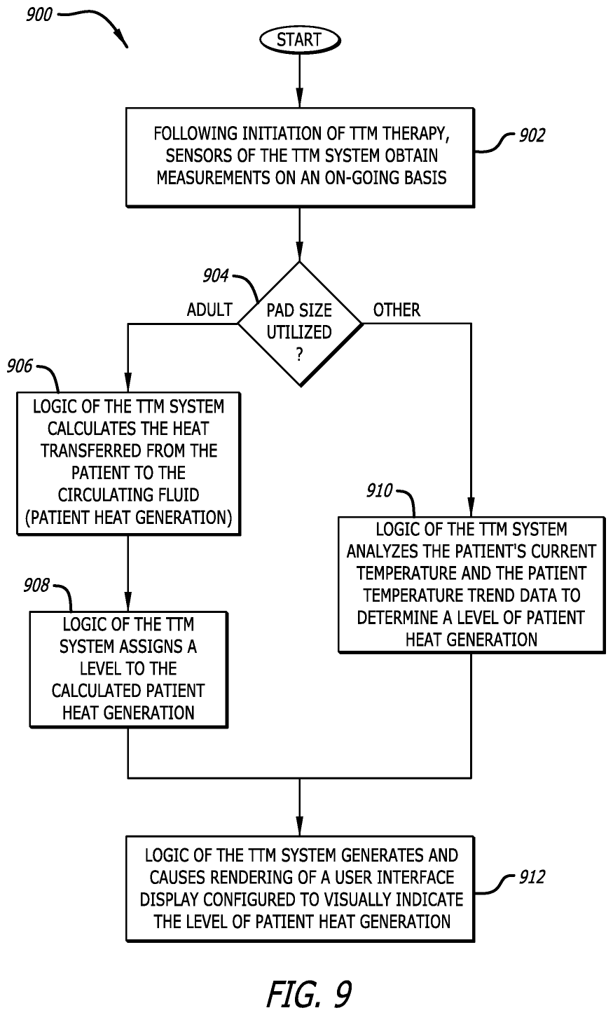

Referring to FIG. 9, a flowchart illustrating a detailed exemplary method of performing patient heat generation detection with the TTM system of FIG. 1 during TTM therapy is shown according to some embodiments. Each block illustrated in FIG. 9 represents an operation performed in the method 900 of performing patient heat generation detection with the TTM system 100. Herein, the method 900 starts with the initiation of a TTM therapy, e.g., in response to receive user input. As discussed above, the TTM therapy may be directed at cooling a patient or rewarming a patient from a state of hypothermia. Following the initiation of TTM therapy, sensors of the TTM system 100 obtain various measurements pertaining to the TTM therapy on an ongoing basis (block 902). As the TTM therapy is being provided by the TTM system 100, the heat generation logic 214 is activated to process the measurements obtained by the sensors and determine a level of patient heat generation. However, in some embodiments, the manner in which patient heat generation is determined (e.g., the one or more analyses, determinations and/or calculations utilized) is dependent on a size of the pads 104 utilized for provision of the therapy. Thus, in such embodiments, the heat generation logic 214 makes a determination as to pad size utilized (block 904). In some embodiments, the determination may be an analysis of user input corresponding to selection of a pad size. However, in other embodiments, coupling of the pads 104 to the control module 102 via fluid delivery lines automatically may provide an indication to the control module 102 as to the pad size (e.g., an electronic signal indicating the pads 104 are coupled may be based to the control module 102 with a product identifier, which may be used by the control module 102 to look up the pad size corresponding to the product identifier).

When the pad size corresponds to a first size (e.g., an adult size), logic of the TTM system 100 calculates the heat transferred from the patient to the circulating fluid (block 906). Subsequently, the logic of the TTM system 100 assigns a level to the calculated patient heat generation (e.g., such as a value of kcal/hour) to one or more predetermined thresholds, where each threshold corresponds to a different level of patient heat generation (block 908). Exemplary calculations performed when the first size pad is used are discussed above with respect to Equations 1-2.

When the pad size corresponds to a second size (e.g., a pediatric or neonate size), logic of the TTM system 100 analyzes the patient's current temperature (optional) and the patient temperature trend data to determine a level of patient heat generation (block 910). Exemplary determinations and analyses performed when a pediatric or neonate size pad is used are discussed above. Although block 910 of FIG. 9 illustrates that the logic of the TTM system analyzes both the patient's current temperature and the patient temperature trend data to determine a level of patient heat generation, utilization of the patient's current temperature is optional (especially with pads sized for neonatal patients). In such an instance, a level of patient heat generation may not be determined but instead the patient temperature trend may be displayed in place of a level of patient heat generation (thus modifying block 912 to generate and cause rendering of a user interface display configured to visually indicate the patient temperature trend).

Following either of blocks 908-910, logic of the TTM system generates and causes rendering of a user interface display configured to visually indicate the level of patient heat generation (block 912).

Referring to FIG. 10, an example user interface display directed to patient heat generation employable in conjunction with implementations of the TTM system of FIG. 1 is shown according to some embodiments. The user interface display 1000 (e.g., an interactive screen) may be provided through the user interface 206 and includes a display portion that graphically illustrates temperature-related data. The user interface display 1000 illustrates data including: (i) an indication of the type of TTM therapy being provided and a target temperature (e.g., “Cooling to: 34.5° C.”); (ii) an indication of the current patient temperature (e.g., “Patient: 36.5° C.”); (iii) an indication of the current fluid temperature (e.g., “Water: 19.0° C.”); and (iv) an indication of the patient heat generation level (e.g., graphical display including a shaded scale having a first third shaded, and the text “baseline”, indicating the patient heat generation level is at “baseline” or the lowest level). It should be understood that the illustrations provided in FIG. 10 are merely one implementation of illustrating such data and the disclosure is not intended to be limited by such.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A patient temperature control system, comprising:
a heat exchange system configured to heat or cool a fluid;
a circulating pump configured to circulate the fluid through the heat exchanger and at least one interconnectable pad;
a patient temperature sensor; and
a control module including one or more fluid temperature sensors, a volumetric flow rate sensor, one or more processors and a non-transitory computer-readable medium having stored thereon logic that, when executed by the one or more processors, causes operations including:
initiating a targeted temperature management (TTM) therapy;
obtaining fluid temperature measurements from the one or more fluid temperature sensors, the fluid temperature measurements comprising a first temperature when the fluid is flowing to the at least one interconnectable pad and a second temperature when the fluid is returning from the at least one interconnectable pad;
obtaining a measurement of volumetric flow rate of the fluid during circulation from the volumetric flow rate sensor;
obtaining one or more measurements of patient temperature from the patient temperature sensor;
determining a level of patient heat generation based on one or more of the fluid temperature measurements, the measurement of volumetric flow rate, or the one or more measurements of patient temperature; and
transmitting via one or more wireless signals, data indicating the level of patient heat generation to a network device.

2. The system of claim 1, wherein the control module is configured to receive return signals from the network device, the return signals employable by the control module to modify the TTM therapy.

3. The system of claim 1, wherein determining the level of patient heat generation includes calculating an amount of heat transfer between the fluid and the patient based on a difference between the first temperature and the second temperature, the volumetric flow rate of the fluid, and a heat capacity of the fluid.

4. The system of claim 1, wherein the logic, when executed by the one or more processors, determines the level of patient heat generation based on the obtained one or more of measurements of patient temperature in accordance with a predetermined rule set.

5. The system of claim 1, wherein the control module is configured to receive user input corresponding to a selection of a size of the at least one interconnectable pad utilized during the TTM therapy, and wherein the logic, when executed by the one or more processors, determines the level of patient heat generation based at least in part on the user input.

6. The system of claim 1, wherein the control module is configured to receive a signal following coupling of the at least one interconnectable pad with the control module employable to identify a size of the at least one interconnectable pad.

7. The system of claim 1, wherein the logic, when executed by the one or more processors, causes further operations including generating a user interface display configured to visually indicate the level of patient heat generation.

8. The system of claim 7, wherein the user interface display is rendered on a display screen of the control module and includes a time-based graphic providing an indication of patient heat generation.

9. The system of claim 8, wherein the time-based graphic provides the indication of patient generation throughout the duration of the TTM therapy following a wait-period following initiation of the TTM therapy.

10. The system of claim 7, wherein the user interface is rendered on a display screen of the network device.

11. The system of claim 1, wherein the level of patient heat generation is utilized as feedback to the logic of the control module and employable to generate one or more instructions to modify operation of the heat exchange system.

12. A method, comprising:
initiating a targeted temperature management (TTM) therapy with a patient temperature control system including a heat exchange system configured to heat or cool a fluid and a circulating pump configured to circulate the fluid through the heat exchanger and at least one interconnectable pad;
obtaining fluid temperature measurements from one or more fluid temperature sensors, the fluid temperature measurements comprising a first temperature when the fluid is flowing to the at least one interconnectable pad and a second temperature when the fluid is returning from the at least one interconnectable pad;
obtaining a measurement of volumetric flow rate of the fluid during circulation from the volumetric flow rate sensor;
obtaining one or more measurements of patient temperature from a patient temperature sensor;
determining a level of patient heat generation based on one or more of the fluid temperature measurements, the measurement of volumetric flow rate, and the one or more measurements of patient temperature; and
transmitting, via one or more wireless signals, data indicating the level of patient heat generation to a network device.

13. The method of claim 12, wherein the patient temperature control system includes a control module including the one or more fluid temperature sensors, the volumetric flow rate sensor, one or more processors and a non-transitory computer-readable medium having stored thereon logic that is executable by the one or more processors.

14. The method of claim 12, wherein the patient temperature control system is configured to receive return signals from the network device, the return signals employable by the patient temperature control system to modify the TTM therapy.

15. The method of claim 12, wherein determining the level of patient heat generation is based at least in part on the obtained measurements of fluid temperature and volumetric flow rate.

16. The method of claim 15, wherein determining the level of patient heat generation includes calculating an amount of heat transfer between the fluid and the patient based on a difference between the first temperature and the second temperature, the volumetric flow rate of the fluid, and a heat capacity of the fluid.

17. The method of claim 12, wherein determining the level of patient heat generation is based on the obtained one or more of measurements patient temperature in accordance with one or more predetermined rule sets.

18. The method of claim 12, further comprising receiving user input, via the patient temperature control system, corresponding to a selection of a size of the at least one interconnectable pad utilized during the TTM therapy, and wherein determining the level of patient heat generation is based at least in part on the user input.

19. The method of claim 12, further comprising generating a user interface display configured to visually indicate the level of patient heat generation via a time-based graphic.

20. The method of claim 19, further comprising causing rendering of the user interface to be rendered on a display screen of the network device.
